(19) United States
(12) Patent Application Publication
(10) Pub. No.: US 2008/0286410 A1
(43) Pub. Date: Nov. 20, 2008

(54) Production of Resistant Starch Product

(76) Inventors: Patricia A. Richmond, Mount Zion, IL (US); Eric A. Marion, Decatur, IL (US); Thomas Eilers, Saraland, AL (US); Annette Evans, Decatur, IL (US); Xian-Zhong Han, Decatur, IL (US); Shakeel Ahmed, Forsyth, IL (US); Donald W. Harris, Springfield, IL (US)

Correspondence Address: WILLIAMS, MORGAN & AMERSON, P.C., 10333 RICHMOND, SUITE 1100, HOUSTON, TX, 77042, US

(21) Appl. No.: 11/970,626

(22) Filed: Jan. 8, 2008

Related U.S. Application Data
(60) Provisional application No. 60/893,160, filed on Mar. 6, 2007.

(51) Int. Cl. A21D 2/08; C12P 19/16; C12P 19/18; C08B 30/12

(52) U.S. Cl. 426/7; 426/28; 426/549; 435/98; 435/97; 127/32

(57) ABSTRACT

A process for producing a starch comprises treating a feed starch that comprises amylopectin with glucanotransferase to produce a chain-extended starch, treating the chain-extended starch with a debranching enzyme to produce a starch product that comprises amylose fragments, crystallizing at least part of the starch product, heating the starch product in the presence of moisture, treating the starch product with alpha-amylase, and washing the starch product to remove at least some non-crystallized starch. The product of this process has a relatively high total dietary fiber content.

This application claims priority from U.S. provisional patent application Ser. No. 60/893,160, filed on Mar. 6, 2007, which is incorporated herein by reference.

### BACKGROUND OF THE INVENTION

Starch comprises two polysaccharides: amylose and amylopectin. Amylose is a generally linear polymer that comprises glucose units connected by alpha 1-4 glycosidic linkages. Amylopectin is a branched polymer in which many of the glucose units are connected by alpha 1-4 glycosidic linkages, but some are connected by alpha 1-6 glycosidic linkages.

Alpha-amylase is an enzyme that is present in the human body and which hydrolyzes alpha 1-4 linkages in starch, thus leading to digestion of the starch. In certain situations it is desirable to produce starch that resists hydrolysis by alpha-amylase, for example to decrease the caloric content of the starch, or to increase its dietary fiber content. However, attempts to produce such starch in the past have suffered from one or more problems, such as high cost.

Amylase-resistant starch is usually produced from high-amylose starch, which is often expensive. There is a need for improved processes for producing starch with a high content of amylose that is suitable for production of alpha-amylase resistant starch.

### SUMMARY OF THE INVENTION

One embodiment of the invention is a process for producing a starch product that comprises (a) treating a feed starch with glucanotransferase to produce a chain-extended starch; (b) treating the chain-extended starch with a debranching enzyme to produce a starch product that comprises amylose fragments; (c) crystallizing at least part of the starch product; (d) heating the starch product in the presence of moisture; (e) treating the starch product with alpha-amylase; and (f) washing the starch product to remove at least some non-crystallized starch. The process can also comprise recovering the remaining starch product after it has been washed. In some embodiments of the process, the feed starch is heated to at least partially gelatinize it prior to treatment with glucanotransferase.

In some embodiments of the invention, at least about 38% by weight of the starch product comprises amylose fragments that have a degree of polymerization (DP) of at least about 35. The process can optionally further include recovering the amylose fragments. As another option, the process can include membrane filtering a solution or dispersion of the starch product to increase the concentration of amylose fragments that have a degree of polymerization (DP) of at least about 35.

Another embodiment of the present invention is a process for producing a starch product that comprises treating a feed starch with glucanotransferase to produce a chain-extended starch; treating the chain-extended starch with a debranching enzyme to produce a starch product that comprises amylose fragments; crystallizing at least part of the starch product; heating the starch product in the presence of moisture; and washing the starch product to remove at least some non-crystallized starch. Other than the absence of treatment with alpha-amylase, various embodiments of this process can be similar to or the same as those of the above-described process.

Another embodiment of the present invention is a starch product produced by any of the above-described processes. In some embodiments of the invention, at least about 40% by weight of the amylose fragments have a degree of polymerization (DP) of at least about 35. If the process used to make the starch product includes membrane filtration, then in some embodiments at least about 50% by weight of the amylose fragments have a degree of polymerization (DP) of at least about 35. In some instances the starch product has a peak melting temperature of greater than about 105° C.

Another embodiment of the invention is a food product that contains the above-described starch product.

### DESCRIPTION OF SPECIFIC EMBODIMENTS

One embodiment of the present invention is a process of producing starch having a relatively high content of amylose. This process includes treating a feed starch that comprises amylopectin with glucanotransferase to extend at least some of the starch chains, and treating the chain-extended starch with a debranching enzyme to produce amylose fragments. These amylose fragments can then be crystallized to produce a resistant starch product.

Ordinary dent corn starch can be debranched enzymatically to give short chain amylose fragments, but since the amylopectin component of the starch is usually composed of relatively short branched chains, the product contains too few of the longer chain lengths that are needed for enzyme resistance. Debranched dent corn starch that has not been modified with a glucanotransferase typically contains less than 35% of the DP35 and higher chain lengths (i.e., starch molecules having a degree of polymerization of at least 35) and therefore does not have the thermal stability needed for a resistant starch. In addition, the debranched dent starch contains a fraction of long chain lengths from amylose as well as short chains from amylopectin. This combination of heterogeneous chain lengths is not optimal for crystallization and amylase resistance.

The feed starch used in the present process can come from a variety of sources, including dent corn, waxy corn, high amylose ae genetic corn (ae is the name of a genetic mutation commonly known by corn breeders and is short for “amylose extender”), potato, tapioca, rice, pea, wheat, waxy wheat, as well as purified amylose from these starches, and alpha-1,4 glucans produced according to patent application WO 00/14249, which is incorporated herein by reference, and combinations of two or more of these starch sources. Chemically modified starches, such as hydroxypropyl starches, starch adipates, acetylated starches, and phosphorylated starches, can also be used in the present invention. For example, suitable chemically modified starches include, but are not limited to, crosslinked starches, acetylated and organically esterified starches, hydroxyethylated and hydroxypropylated starches, phosphorylated and inorganically esterified starches, cationic, anionic, nonionic, and zwitterionic starches, and succinate and substituted succinate derivatives of starch. Such modifications are known in the art, for example in Modified Starches: Properties and Uses, Ed. Wurzburg, CRC Press, Inc., Florida (1986). Other suitable modifications and methods are disclosed in U.S. Pat. Nos. 4,626,288, 2,613,206 and 2,661,349, which are incorporated herein by reference.

If the feed starch is a waxy starch, it can be at least partially debranched by treatment with a debranching enzyme prior to treatment with glucanotransferase. Suitable debranching enzymes for this purpose include pullulanase and isoamylase. This provides a source of fragments that will be transferred by the glucanotransferase to the amylopectin non-reducing ends, resulting in longer branched chains.

4-α-glucanotransferase [2.4.1.25] is an enzyme that catalyzes the transfer of a segment of a 1,4-alpha-D-glucan to a new position in an acceptor, which can be glucose or another 1,4-alpha-D-glucan. Glucanotransferase will catalyze the transfer of a maltosyl moiety to a maltotriose acceptor, releasing glucose. The glucose released can be used as a measurement of enzyme activity.

A suitable assay for determining glucanotransferase activity is as follows. In this assay, maltotriose is used as both substrate and acceptor molecule. Glucose is released in this reaction and can be measured after a modified version of the common glucose oxidase/peroxidase assay. (Werner, W. et al (1970) Z. Analyt. Chem. 252:224.) GOD-Perid solution can be obtained from a Glucose Release Kit from WAKO, or can be prepared with 65 mM sodium phosphate, pH 7 including 0.4 g/l glucose oxidase (Sigma G6125 or G7773), 0.013 g/l HRP (Sigma P8125), and 0.65 g/l ABTS (Calbiochem #194430). A 0.04 N NaOH solution is also used. The substrate solution is 1% maltotriose (0.1 g maltotriose in 10 ml of 50 mM phosphate buffer at pH 6.0).

### Standard Curve:

Glucose solution: weight out 0.1806 g glucose into 500 ml MQ H₂O.

### Dilutions for Standard Curve:

| Concentration | | μL glucose solution | μL MQ water |
|---|---|---|---|
| 0.01 | μmol | 5 | 495 |
| 0.05 | μmol | 25 | 475 |
| 0.1 | μmol | 50 | 450 |
| 0.25 | μmol | 125 | 375 |
| 0.5 | μmol | 250 | 250 |

120 μl of the substrate solution is pre-incubated at a selected temperature, e.g. 60° C., for 10 minutes. 20 μl of enzyme solution are added to the substrate solution and the reaction mixture is incubated at 600 for 10 minutes. The reaction is stopped by the addition of 20 μl of 0.04N NaOH. 20 μl is then transferred to a 96 well microtiter plate and 230 μl GOD-Perid solution is added. After 30 minutes at room temperature, the absorbance is measured at 420 nm. The enzyme activity is calculated relative to the standard curve of glucose in the range of 0-0.5 gmol glucose. One unit (U) of activity is defined as the amount of enzyme that liberates 1 μmol glucose/minute.

In some embodiments of the process, the glucanotransferase is used in a dosage of about 1-18,000 GTU per gram of feed starch. In other embodiments, the glucanotransferase is used in a dosage of about 10-18 GTU per gram of feed starch. Optionally, the glucanotransferase is used in a plurality of dosages that are supplied to the feed starch at separate times.

Treatment of the feed starch with glucanotransferase produces extensions of the chains on the amylopectin molecules. This treatment can be performed, for example, in aqueous solution or suspension at a temperature of about 70-100° C. and a pH of about 5.0-8.5. As a result, the DP35 and higher content of the end product increases to over 38%, or in some cases to over 40%, and the chain lengths are much more uniform, which is indicated by a polydispersity of 2-4, compared to about 8 for debranched dent corn starch. In some embodiments of the invention, the dosage of glucanotransferase can be about 1-15 ml per 100 gram of starch, preferably about 5-12 ml/100 g. The glucanotransferase can be contacted with the starch in a single dose, or split into multiple doses. In one embodiment of the invention, the total dosage is split into three portions which are provided at separate times (for example, three separate doses of 2.5 ml/100 g each), with at least one hour between each. In some embodiments, the reaction temperature can be from about 75-85° C., and the reaction time can be less than about 8 hours, preferably less than about 6 hours.

Optionally, an additional starch-based material can be added to the chain-extended starch prior to debranching. For example, a maltodextrin can be added.

The resulting chain-extended starch can then be treated with a debranching enzyme, such as isoamylase or pullulanase, for example at a temperature of about 30-60° C. and a pH of about 4.0-5.0 to produce amylose fragments having desirable lengths. In some embodiments of the process, the debranching enzyme is used in a dosage of at least about 0.1 ml per gram of chain extended starch. In other embodiment, the debranching enzyme is used in a dosage of at least about at least about 1.0 ml per gram of chain extended starch. In certain embodiments of the invention, a dosage of isoamylase of about 1-10 mg per g of starch is used, preferably about 1-5 mg/g.

The DP35 and higher content can be enriched to over 50% by fractionation by microfiltration at an elevated temperature, such as about 60-120° C., more typically about 60-90° C., and even more typically 70-85° C. The debranched, glucanotransferase-treated, starch product after microfiltration can have a peak melting temperature greater than about 105° C., and can contain at least about 80% by weight resistant starch after heating in water to about 98° C.

Optionally, the debranched starch produced in step (b) is gelatinized in a jet cooker to solubilize the starch, and then is cooled to about 20-90° C. to crystallize.

Optionally, the product starch can be heat treated in the presence of moisture at a temperature of at least about 90° C., or in some embodiments at least about 98° C. In some embodiments of the process, in step (d) the starch product is heated to about 100-150° C. at a moisture content of about 15-35% by weight. In other embodiments, in step (d) the starch product is heated to about 120-130° C. at a moisture content of about 22-26% by weight. This heat-moisture treatment can increase the total dietary fiber (TDF) content and/or the resistant starch (RS) content of the starch product in some instances. For example, in some embodiments, the starch product has a total dietary fiber (TDF) content of at least about 10% by weight before the heat moisture treatment in step (d), or, in some instances, a TDF content greater than about 30% by weight before the heat moisture treatment in step (d). In some embodiments, the starch product has a TDF content of at least about 50% by weight after the heat moisture treatment of step (d), or, in some cases, a TDF content of greater than about 75% by weight after the heat moisture treatment of step (d). In some embodiments, the starch product has a resistant starch (RS) content of at least 40% by weight before the heat moisture treatment of step (d), and, in some cases, a RS content greater than about 80% by weight after the heat moisture treatment of step (d).

The heat moisture treatment can increase the TDF (AOAC 991.43) of the starch from about 15-35% to about 75-80% in some embodiments of the invention.

In one embodiment of the process, the feed starch is slurried in water at 15% solids and the pH is adjusted to 5.5 with dilute NaOH. The slurry is placed in an autoclave and heated to 140° C. for 30 minutes. After cooling to 85° C. and adjusting the pH to 5.5, glucanotransferase is added and allowed to react for 24 hours. The enzyme is deactivated by reducing the pH to below 3.0. The starch is redispersed by heating to 140° C. for one hour and then cooled to 45° C., and the pH is adjusted to 4.5. Isoamylase is added and allowed to react for 18-24 hours. The mixture is heated to 85° C. for one hour to deactivate the enzyme. If necessary, the product can be treated again with isoamylase by repeating the 140° C. heating and enzyme treatment at 45° C. and pH 4.5. The product can then be fractionated to increase the content of longer chain components. This can be carried out, for example, by microfiltration or ultrafiltration of the crystallized debranched product at a temperature of at least about 80° C. using a ceramic membrane with a pore size of about 0.45 microns. After collecting 1.5 to 2.5 volumes of permeate relative to the volume of the starting slurry, while maintaining the volume of the retentate by addition of deionized water, the product is isolated by concentrating and spray drying or by centrifuging and oven drying the retentate.

In another embodiment of the process, a starch product that comprises a substantial percentage of resistant starch can be produced by (a) treating a feed starch with glucanotransferase to produce a chain-extended starch; (b) treating the chain-extended starch with a debranching enzyme to produce a starch product that comprises amylose fragments; (c) crystallizing at least part of the starch product; (d) heating the starch product in the presence of moisture; (e) treating the starch product with alpha-amylase; and (f) washing the starch product. The remaining starch product can be recovered after it has been washed (i.e., after at least some of the non-crystallized components, and preferably the majority of such components, are removed by the washing). In many cases, the feed starch is heated to at least partially gelatinize it prior to treatment with glucanotransferase.

The heat/moisture treatment in step (d) helps to increase the percentage of total dietary fiber (TDF) and resistant starch (RS) in the starch product. Resistant starch content was analyzed using the method of Englyst et al. (Eur. J. Clinical Nut. (1992) 46 (Suppl. 2), S33-S50, “Classification and Measurement of Nutritionally Important Starch Fractions”). (All references in this patent to a percentage of resistant starch in a material are as determined by the Englyst assay.)

As an example of suitable conditions for this step, the starch product can be heated to about 120-150° C. with a beginning moisture content of about 20-35% by weight, for a time of about 1-12 hours. In some embodiments of the invention, the starch product is heated to about 125-135° C. with a beginning moisture content of about 25-27% by weight. At the conclusion of this step, in some embodiments of the process, the starch product will have a TDF content of about 70-80% by weight, a DSC enthalpy of about 22 Joules/gram, and good thermal stability.

The additional steps of treating the starch product with alpha-amylase and washing can increase the TDF content by removing at least some non-crystallized starch. The non-crystallized material tends to be more susceptible to degradation by amylase, and therefore its removal will usually boost the TDF and RS values of the product. In some embodiments, at the conclusion of these additional steps, at least about 50% by weight of the recovered starch product is oligomers having a degree of polymerization (DP) from about 24-100 (inclusive), and in some cases, at least about 75% by weight of the recovered starch product has a DP from about 24-100. In some embodiments, the recovered starch product has an enthalpy as measured by differential scanning calorimetry of at least about 20 Joules/gram. In some embodiments, the recovered starch product has a peak melting temperature of greater than about 105° C., a TDF content of at least about 85% by weight, and an enthalpy as measured by differential scanning calorimeter of at least about 27 Joules/gram. In certain embodiments, the starch product has a TDF value of 85-90% by weight and a DSC enthalpy of about 28 Joules/gram.

One advantage of the process is that it can produce a high TDF starch product from dent corn, and does not require a feed starch with unusually high amylose content. This makes the process more economical.

The product produced by the process contains a high percentage of amylose that is resistant to alpha-amylase. The resistant starch can be added to a number of food products to reduce their caloric density and glycemic index, and increase dietary fiber and probiotic effect in the colon.

Starch produced by this process can be used as a bulking agent or flour substitute in foods, such as reduced calorie baked goods. The starch is also useful for dietary fiber fortification in foods. Specific examples of foods in which the starch can be used include bread, cakes, cookies, crackers, extruded snacks, soups, frozen desserts, fried foods, pasta products, potato products, rice products, corn products, wheat products, dairy products, nutritional bars, food for diabetics, and beverages.

The starch product, at least in some embodiments, is thermally stable in water at a temperature of at least about 90° C., or in some cases at least about 100° C., allowing it to be used in food products that will be processed at high temperature and moisture conditions.

In some embodiments, the starch product has a crystal morphology (as determined by wide angle X-ray diffraction techniques) of A form, B form, or a combination thereof. In other words, the product can comprise 100% A form crystals, 100% B form crystals, or any blend of the two forms.

Certain embodiments of the invention are described in the following example.

### Example 1

### Preparation of Heat/Moisture Treated Resistant Starch

250 lb of regular dent corn starch and 1420 lb water were added to a vessel to give a 15% starch slurry. The starch slurry was jet cooked at approximately 149° C. at a feed rate of approximately 2.0 gpm and the resulting paste was flashed into a tank and maintained at approximately 88° C., with agitation. Into the dent corn starch paste as it entered the tank, was injected a total of approximately 8,000 GTU/lb starch of 4-α-glucanotransferase enzyme (obtained from Novozymes) spread over the entire time period the paste was pumped into the tank. The mixture was allowed to react for 3 hr at 88° C. with agitation. Dilute sulfuric acid was added to adjust the pH to 3.8-3.9 and the reactor contents were cooled rapidly to approximately 55° C. by pumping through a heat exchanger into an agitated tank maintained at 55° C. To the slurry was added 0.1 ml/100 g of starch of isoamylase enzyme obtained from Hayashibara Co. and the enzyme was allowed to react 16 hr at 55° C. while maintaining the pH at 3.8-3.9. The slurry was then jet cooked at approximately 149° C. and allowed to cool slowly with stirring to 55° C. then held at 55° C. overnight to promote crystal formation. The slurry was then dewatered on a basket centrifuge and dried overnight in a tray dryer to approximately 10% moisture content. The resistant starch product was ground to pass through a US #40 mesh sieve. To 55 lb of resistant starch from the above process, with agitation, was added sufficient water to give 25% total water content. The starch cake was placed in a steam jacketed Littleford Reactor and heated with agitation in a nitrogen atmosphere at approximately 126° C. for 2 hr. The mixture was then cooled and taken from the Littleford Reactor and tray dried to approximately 10% moisture content. The resulting heat/moisture treated resistant starch product was ground to pass through a US #40 mesh sieve.

Preparation of High TDF α-Amylase Enzyme Treated Resistant Starch Product:

To 200 g of the heat/moisture treated resistant starch above was added 800 g water and the pH was adjusted to approximately 8.2 by drop-wise addition of dilute soda ash solution. The slurry was heated to 95° C. in a 2-liter round bottom flask and 10 ml of alpha-amylase enzyme (3,000 units/ml) obtained from a Megazyme Total Dietary Fibre kit was added and the slurry was maintained at 95° C. by immersing the flask and contents in a controlled temperature water bath. After 1 hr. of reacting at 95° C., half of the slurry was removed and filtered on a Buchner funnel then washed with 300 ml deionized water. The wet cake was dried in a 50° C. oven overnight. The dried product was ground to pass through a US #40 mesh sieve. The yield of this dry resistant starch product was calculated to be 77.8%. After 2 hr. of reacting the remaining half of the slurry was filtered, washed, dried, ground and sieved as above. The yield of this dry resistant starch product was calculated to be 78.6%. Analysis of the alpha-amylase treated heat/moisture treated resistant starch products is given in Table 1 below.

TABLE 1

| | | | | DSC Analysis | | |
|---|---|---|---|---|---|---|
| | Yield, | TDF, | | Onset, | Peak, | Enthalpy, |
| Description | % | % | RS** | ° C. | ° C. | J/g |
| α-amylase | 77.8 | 90.0 | 97.2 | 105 | 117 | 27.9 |
| treated 1 hr | | | | | | |
| α-amylase | 78.6 | 89.2 | 96.6 | 105 | 117 | 28.8 |
| treated 2 hr | | | | | | |
| for cookie | 74.9 | 85.4 | 96.6 | 104 | 116 | 27.4 |
| application* | | | | | | |

*Prepared used Termamyl SC α-amylase enzyme.
**Englyst assay for resistant starch.

### Example 2

### Preparation of Resistant Starch

250 lb of regular dent corn starch was added to sufficient water to give a 17.5% starch slurry. The starch slurry was jet cooked at approximately 149° C. at a feed rate of approximately 2.0 gpm and the resulting paste was flashed into a tank and maintained at approximately 88° C., with agitation. 4-α-glucanotransferase enzyme (obtained from Novozymes) was added to the dent corn starch paste as it entered the tank. A total of approximately 8,000 GTU/(lb starch) was added over the entire time period the paste was pumped into the tank. The mixture was allowed to react for 3 hr at 88° C. with agitation. Dilute sulfuric acid was added to adjust the pH to 3.8-3.9 and the reactor contents were cooled rapidly to approximately 55° C. by pumping through a heat exchanger into an agitated tank maintained at 55° C. To the slurry was added 0.1 ml/100 g of starch of isoamylase enzyme obtained from Hayashibara Co. and the enzyme was allowed to react 16 hr at 55° C. while maintaining the pH at 3.8-3.9. The slurry was then jet cooked at approximately 148° C. and allowed to cool slowly with stirring to 55° C. then held at 55° C. overnight to promote crystal formation. The pH of the slurry was then adjusted to 6.0-7.0 and the slurry was then dewatered on a basket centrifuge and dried overnight in a tray dryer to approximately 10% moisture content. The resistant starch product was ground to pass through a US #40 mesh sieve. The starch was added to a jacketed high intensity mixer and water was added through a spray nozzle. Sufficient water was added to 60 lbs of resistant starch from the above process, with agitation, to give 24-25% total water content. The mixer was sealed up and starch was heated by steam in the jacket of the mixer to a temperature of 129° C. The starch was held at 24-25% moisture and 129° C. for 2 hours. The mixture was then dried to approximately 10% moisture content in the high intensity mixer. The resulting heat/moisture treated resistant starch product was ground to pass through a US #40 mesh sieve. Analysis of the resistant starch prepared in this example, both before and after heat moisture treatment is presented below:

TABLE 2
Analysis of the Resistant Starch Before Heat Moisture Treatment

| | DSC Analysis | | | % DP Greater |
|---|---|---|---|---|
| % TDF | Onset (° C.) | Peak (° C.) | Enthalpy (J/g) | than 37 |
| 22.0 | 90.5 | 112.8 | 20.5 | 71.8 |

TABLE 3
Analysis of the Resistant Starch After Heat Moisture Treatment

| | DSC Analysis | | |
|---|---|---|---|
| % TDF | Onset (° C.) | Peak (° C.) | Enthalpy (J/g) |
| 80.7 | 104.2 | 117.1 | 24.1 |

### Example 3

### Preparation of Material that is Mostly ‘A’ Type Crystals

250 lbs of regular dent corn starch was added to sufficient water to produce a 17.5% starch slurry. The starch slurry was jet cooked at approximately 150° C. at a feed rate of approximately 2 gpm and the resulting paste was flashed into a tank maintained at approximately 88° C., with agitation. Into the dent corn starch paste was added 2400 GTU/lb starch of 4-α-glucanotransferase enzyme (obtained from Novozymes) spread over the entire time period the paste was pumped into the tank. The mixture was allowed to react for 3 hr at 88° C. with agitation. Dilute sulfuric acid was added to adjust the pH to 3.8-3.9 and the reactor contents were cooled rapidly to approximately 55° C. by pumping through a heat exchanger into an agitated tank maintained at 55° C. To the slurry was added 0.1 ml/100 g of starch of isoamylase enzyme obtained from Hayashibara Co. and the enzyme was allowed to react for 16 hrs at 55° C. while maintaining the pH at 3.8-3.9. The slurry was then jet cooked at approximately 149° C. and allowed to slowly cool to 70° C. The slurry was taken from the reactor and placed into a 3000 mL agitated glass reactor in a water bath set at 65° C. The slurry was allowed to agitate in the reactor for 96 hrs at 65° C. The material was dewatered on a Buchner funnel and dried overnight in a tray dryer. A heat moisture treatment was subsequently completed on the starch in the laboratory in which the starch was subjected to 135° C. at 25% moisture for three hours. Analysis of the material is presented below.

### Example 4

### Preparation of Material that is Completely ‘B’ Type Crystals

250 lbs of regular dent corn starch was added to sufficient to produce a 17.5% starch slurry. The starch slurry was jet cooked at approximately 147° C. at a feed rate of approximately 2 gpm and the resulting paste was flashed into a tank maintained at approximately 88° C., with agitation. Into the dent corn starch paste was added 8000 GTU/lb starch of 4-α-glucanotransferase enzyme (obtained from Novozymes) spread over the entire time period the paste was pumped into the tank. The mixture was allowed to react for 2 hr at 88° C. with agitation. Dilute sulfuric acid was added to adjust the pH to 3.8-3.9 and the reactor contents were cooled rapidly to approximately 55° C. by pumping through a heat exchanger into an agitated tank maintained at 55° C. To the slurry was added 0.1 ml/100 g of starch of isoamylase enzyme obtained from Hayashibara Co. and the enzyme was allowed to react for 16 hrs at 55° C. while maintaining the pH at 3.8-3.9. The slurry was then jet cooked at approximately 151° C. and allowed to slowly cool to 55° C. then held at 55° C. overnight to promote crystal formation. The slurry was then dewatered on a basket centrifuge and dried overnight in a tray dryer to approximately 10% moisture content. The resistant starch product was ground to pass through a US #40 mesh sieve. A heat moisture treatment was completed on 10 lbs of the dried material in a Littleford DVT-22 at 24% moisture and 126° C. for 2 hrs. Analysis of the material is presented below.

TABLE 4

| | Before Heat Moisture Treatment(HMT) | | | | | | |
|---|---|---|---|---|---|---|---|
| | % | % A | | DSC | DSC | DSC | After |
| | crystal- | type/% | % | Onset | Peak | Enthalpy | HMT |
| Description | linity | B type | TDF | (° C.) | (° C.) | J/g | % TDF |
| Process to | 67 | 80/20 | 18.7 | 105 | 133 | 23.0 | 75.8 |
| form majority | | | | | | | |
| A type | | | | | | | |
| Process to | 60 | 100/0 | 18.7 | 88 | 113 | 23.6 | 84.9 |
| form B type | | | | | | | |

### Example 5

Evaluation of Different Treated Heat/Moisture Treatment Conditions

Material was produced in a similar fashion to previous examples through the step where the material was centrifuged and dried (prior to heat moisture treatment). The dried material was subjected to heat moisture treatments at different moisture levels and temperatures. During the experiments, nine pounds of starch was added to an agitated Littleford DVT-22. Water was added through a spray nozzle to reach the desired moisture target. The Littleford DVT-22 was closed up and the starch was heated by steam on the Littleford jacket to the desired temperature. The material was kept at the desired temperature for 2 hrs after which time the Littleford was opened up and the starch was allowed to cool. % TDF and DSC analysis was performed on the starch and is presented below in Table 5. Additionally, crystallinity and crystal types were determined for select lots.

TABLE 5

| | | | | DSC Analysis | | | % | |
|---|---|---|---|---|---|---|---|---|
| | Temp | Moisture | % | Onset | Peak | Enthalpy | crystal- | % A type/ |
| Run | (° C.) | (%) | TDF | (° C.) | (° C.) | J/g | linity | % B type |
| 1 | 125 | 17.9 | 63.1 | 93 | 119 | 24.4 | 66 | 75/25 |
| 2 | 120 | 20 | 63.4 | 97 | 117 | 21.7 | | |
| 3 | 130 | 20 | 69 | 100 | 121 | 24.8 | | |
| 4 | 117.9 | 25 | 66.7 | 103 | 117 | 22.9 | 69 | 45/55 |
| 5 | 125 | 25 | 77.5 | 108 | 123 | 27.1 | 65 | 60/40 |
| 6 | 125 | 25 | 78.3 | 110 | 122 | 25.0 | | |
| 7 | 125 | 25 | 78.3 | 109 | 121 | 23.0 | | |
| 8 | 132.07 | 25 | 74.1 | 112 | 139 | 22.4 | 78 | 95/5 |
| 9 | 120 | 30 | 66.7 | 108 | 135 | 23.4 | | |
| 10 | 130 | 30 | 59 | 113 | 139 | 22.2 | | |
| 11 | 125 | 32.07 | 53.7 | 110 | 138 | 24.2 | 85 | 100/0 |
| 12 | 125 | 25 | 75.2 | 109 | 122 | 23.0 | | |

### Example 6

### Results of Using Microfiltration to Recover Crystalline Product

Microfiltration was carried out in a system comprising a reservoir with a heating jacket connected to a recirculation pump and a housing containing a Millipore 0.45 micron ceramic membrane. The jacket was heated with a circulation oil bath and the membrane housing was heated with an electric heating tape. The membrane housing was generally maintained at 10-15° C. higher than the reservoir temperature to prevent crystallization of debranched material in the membrane.

Glucanotransferase-treated dent starch suspension (1056.9 g at about 5% solids) was diluted with 297 g of deionized water and heated in the microfiltration reservoir with recirculation to 80° C. and held for 1 hour before starting to draw permeate from the membrane housing. As permeate was collected an equal volume of deionized water was added to the reservoir. After 3360 g of permeate was collected, the retentate (1236 g) was withdrawn from the reservoir and allowed to cool in a beaker placed in a refrigerator. The retentate contained 34.1 g of dry solids and the permeate contained 9.0 g of dry solids. The retentate was isolated by dilution of the slurry with formula 3A ethanol, filtering and drying. The molecular weight of the debranched, glucanotransferase-treated starch and the retentate and permeate fractions from microfiltration were analyzed by GPC. The retentate was tested for resistant starch (RS). The results are shown in Table 6.

TABLE 6
Micrfiltration Fractionation of Debranched, Glucanotransferase-Treated
Dent Starch @ 80° C.

| | Permeate | Yield | DP | DP | DP | | | | |
|---|---|---|---|---|---|---|---|---|---|
| Sample | Volume* | wt. % | 37-60 | 60-100 | 100+ | Mw | Mn | DP37+ | % RS |
| starting sample | ** | ** | 25.2 | 13.7 | 0.8 | 5762 | 3708 | 39.7 | |
| retentate | 2.5/l | 73.5 | 35.7 | 15.5 | 0.3 | 6539 | 5083 | 51.5 | 87.2 |
| permeate | 2.5/l | 26.5 | 13.9 | 1.4 | 0 | 3883 | 3022 | 15.2 | |

*“2.2/l” indicates that the sample was washed and that 2.5 liters of permeate was collected per liter of starting sample.

The preceding description of specific embodiments of the invention is not intended to be a list of every possible embodiment of the invention. Persons skilled in the art will recognize that other embodiments would be within the scope of the following claims.

What is claimed is:

1. A process for producing a starch product, comprising:
(a) treating a feed starch with glucanotransferase to produce a chain-extended starch;
(b) treating the chain-extended starch with a debranching enzyme to produce a starch product that comprises amylose fragments;
(c) crystallizing at least part of the starch product;
(d) heating the starch product in the presence of moisture;
(e) treating the starch product with alpha-amylase; and
(f) washing the starch product to remove at least some non-crystallized starch.

2. The process of claim 1, further comprising recovering the remaining starch product after it has been washed.

3. The process of claim 1, wherein the feed starch is from dent corn, waxy corn, high amylose ae genetic corn, potato, tapioca, rice, pea, wheat, waxy wheat, or a combination of two or more thereof.

4. The process of claim 1, wherein the feed starch is heated to at least partially gelatinize the starch prior to treatment with glucanotransferase.

5. The process of claim 1, wherein the feed starch is treated with glucanotransferase in aqueous solution or suspension at a temperature of about 70-100° C. and a pH of about 5.0-8.5.

6. The process of claim 1, wherein the debranching enzyme is isoamylase or pullulanase.

7. The process of claim 1, further comprising membrane filtering a solution or dispersion of the starch product to increase the concentration of amylose fragments that have a degree of polymerization (DP) of at least 35.

8. The process of claim 1, wherein at least about 40% by weight of the amylose fragments have a degree of polymerization (DP) of at least 35.

9. The process of claim 1, wherein at least about 50% by weight of the amylose fragments have a degree of polymerization (DP) of at least 35.

10. The process of claim 1, wherein the glucanotransferase is used in a dosage of about 1-18,000 GTU per gram of feed starch.

11. The process of claim 1, wherein the glucanotransferase is used in a dosage of about 10-18 GTU per gram of feed starch.

12. The process of claim 1, wherein the glucanotransferase is used in a plurality of dosages that are supplied to the feed starch at separate times.

13. The process of claim 1, wherein the debranching enzyme is used in a dosage of at least about 0.1 ml per gram of chain extended starch.

14. The process of claim 13, wherein the debranching enzyme is used in a dosage of at least about at least about 1.0 ml per gram of chain extended starch.

15. The process of claim 1, wherein in step (d) the starch product is heated to about 100-150° C. at a moisture content of about 15-35% by weight.

16. The process of claim 15, wherein in step (d) the starch product is heated to about 120-130° C. at a moisture content of about 22-26% by weight.

17. The process of claim 1, wherein the starch product has a total dietary fiber (TDF) value of at least about 10% by weight before the heat moisture treatment in step (d).

18. The process of claim 17, wherein the starch product has a total dietary fiber (TDF) value greater than about 30% by weight before the heat moisture treatment in step (d).

19. The process of claim 1, wherein the starch product has a total dietary fiber (TDF) value of at least about 50% by weight after the heat moisture treatment of step (d).

20. The process of claim 19, wherein the starch product has a total dietary fiber (TDF) value of greater than about 75% by weight after the heat moisture treatment of step (d).

21. The process of claim 1, wherein the starch product has a resistant starch value of at least 40% by weight before the heat moisture treatment of step (d).

22. The process of claim 1, wherein the starch product has a resistant starch value greater than about 80% by weight after the heat moisture treatment of step (d).

23. The process of claim 1, wherein the debranched starch produced in step (b) is gelatinized in a jet cooker to solubilize the starch, and then is cooled to about 20-90° C. to crystallize.

24. The process of claim 1, wherein the starch product has a crystal morphology of A form, B form, or a combination thereof.

25. The process of claim 1, wherein the feed starch is a waxy starch, and wherein the process further comprises treating the feed starch with a debranching enzyme before the feed starch is treated with glucanotransferase.

26. The process of claim 2, wherein at least about 50% by weight of the recovered starch product has a degree of polymerization (DP) from about 24-100.

27. The process of claim 26, wherein at least about 75% by weight of the recovered starch product has a degree of polymerization (DP) from about 24-100.

28. The process of claim 2, wherein the recovered starch product has a peak melting temperature of greater than about 105° C.

29. The process of claim 2, wherein the recovered starch product is thermally stable in water at temperatures up to at least about 90° C.

30. The process of claim 2, wherein the recovered starch product is thermally stable in water at temperatures up to at least about 100° C.

31. The process of claim 2, wherein the recovered starch product has an enthalpy as measured by differential scanning calorimetry of at least about 20 Joules/gram.

32. A starch product produced by a process comprising:
(a) treating a feed starch with glucanotransferase to produce a chain-extended starch;
(b) treating the chain-extended starch with a debranching enzyme to produce a starch product that comprises amylose fragments;
(c) crystallizing at least part of the starch product;
(d) heating the starch product in the presence of moisture;
(e) treating the starch product with alpha-amylase;
(f) washing the starch product to remove at least some non-crystallized starch; and
(g) recovering the remaining starch product.

33. The starch product of claim 32, wherein the feed starch is from dent corn, waxy corn, high amylose ae genetic corn, potato, tapioca, rice, pea, wheat, waxy wheat, or a combination of two or more thereof.

34. The starch product of claim 32, wherein the feed starch is heated to at least partially gelatinize the starch prior to treatment with glucanotransferase.

35. The starch product of claim 32, wherein the feed starch is treated with glucanotransferase in aqueous solution or suspension at a temperature of about 70-100° C. and a pH of about 5.0-8.5.

36. The starch product of claim 32, wherein the debranching enzyme is isoamylase or pullulanase.

37. The starch product of claim 32, further comprising membrane filtering a solution or dispersion of the starch product to increase the concentration of amylose fragments that have a degree of polymerization (DP) of at least 35.

38. The starch product of claim 32, wherein at least about 40% by weight of the amylose fragments have a degree of polymerization (DP) of at least 35.

39. The starch product of claim 32, wherein at least about 50% by weight of the amylose fragments have a degree of polymerization (DP) of at least 35.

40. The starch product of claim 32, wherein the glucanotransferase is used in a dosage of about 1-18,000 GTU per gram of feed starch.

41. The starch product of claim 32, wherein the glucanotransferase is used in a dosage of about 10-18 GTU per gram of feed starch.

42. The starch product of claim 32, wherein the glucanotransferase is used in a plurality of dosages that are supplied to the feed starch at separate times.

43. The starch product of claim 32, wherein the debranching enzyme is used in a dosage of at least about 0.1 ml per gram of chain extended starch.

44. The starch product of claim 43, wherein the debranching enzyme is used in a dosage of at least about at least about 1.0 ml per gram of chain extended starch.

45. The starch product of claim 32, wherein at least about 50% by weight of the recovered starch product has a degree of polymerization (DP) from about 24-100.

46. The starch product of claim 32, wherein the recovered starch product has a peak melting temperature of greater than about 105° C.

47. The starch product of claim 32, wherein the recovered starch product has an enthalpy as measured by differential scanning calorimetry of at least about 20 Joules/gram.

48. A food product comprising a starch, wherein the starch is produced by a process comprising:
(a) treating a feed starch with glucanotransferase to produce a chain-extended starch;
(b) treating the chain-extended starch with a debranching enzyme to produce a starch product that comprises amylose fragments;
(c) crystallizing at least part of the starch product;
(d) heating the starch product in the presence of moisture;
(e) treating the starch product with alpha-amylase;
(f) washing the starch product to remove at least some non-crystallized starch; and
(g) recovering the remaining starch product.

49. The food product of claim 48, wherein the feed starch is from dent corn, waxy corn, high amylose ae genetic corn, potato, tapioca, rice, pea, wheat, waxy wheat, or a combination of two or more thereof.

50. The food product of claim 48, wherein the feed starch is heated to at least partially gelatinize the starch prior to treatment with glucanotransferase.

51. The food product of claim 48, wherein the feed starch is treated with glucanotransferase in aqueous solution or suspension at a temperature of about 70-100° C. and a pH of about 5.0-8.5.

52. The food product of claim 48, wherein the debranching enzyme is isoamylase or pullulanase.

53. The food product of claim 48, wherein in step (d) the starch product is heated to about 100-150° C. at a moisture content of about 15-35% by weight.

54. The food product of claim 53, wherein in step (d) the starch product is heated to about 120-130° C. at a moisture content of about 22-26% by weight.

55. The food product of claim 48, wherein the starch product has a total dietary fiber (TDF) value of at least about 10% by weight before the heat moisture treatment in step (d).

56. The food product of claim 55, wherein the starch product has a total dietary fiber (TDF) value greater than about 30% by weight before the heat moisture treatment in step (d).

57. The food product of claim 48, wherein the starch product has a total dietary fiber (TDF) value of at least about 50% by weight after the heat moisture treatment of step (d).

58. The food product of claim 57, wherein the starch product has a total dietary fiber (TDF) value of greater than about 75% by weight after the heat moisture treatment of step (d).

59. The food product of claim 48, wherein the starch product has a resistant starch value of at least 40% by weight before the heat moisture treatment of step (d).

60. The food product of claim 48, wherein the starch product has a resistant starch value greater than about 80% by weight after the heat moisture treatment of step (d).

61. The food product of claim 48, wherein the debranched starch produced in step (b) is gelatinized in a jet cooker to solubilize the starch, and then is cooled to about 20-90° C. to crystallize.

62. The food product of claim 48, wherein the starch product has a crystal morphology of A form, B form, or a combination thereof.

63. The food product of claim 48, further comprising membrane filtering a solution or dispersion of the starch product to increase the concentration of amylose fragments that have a degree of polymerization (DP) of at least 35.

64. The food product of claim 48, wherein at least about 40% by weight of the amylose fragments have a degree of polymerization (DP) of at least 35.

65. The food product of claim 48, wherein at least about 50% by weight of the amylose fragments have a degree of polymerization (DP) of at least 35.

66. The food product of claim 48, wherein the glucanotransferase is used in a dosage of about 1-18,000 GTU per g of feed starch.

67. The food product of claim 48, wherein the glucanotransferase is used in a dosage of about 10-18 GTU per grams of feed starch.

68. The food product of claim 48, wherein at least about 50% by weight of the recovered starch product has a degree of polymerization (DP) from about 24-100.

69. The food product of claim 48, wherein the recovered starch product has a peak melting temperature of greater than about 105° C.

70. The food product of claim 48, wherein the recovered starch product has an enthalpy as measured by differential scanning calorimetry of at least about 20 Joules/gram.

71. A process for producing a starch product, comprising:
(a) treating a feed starch with glucanotransferase to produce a chain-extended starch;
(b) treating the chain-extended starch with a debranching enzyme to produce a starch product that comprises amylose fragments;
(c) crystallizing at least part of the starch product;
(d) heating the starch product in the presence of moisture; and
(e) washing the starch product to remove at least some non-crystallized starch.

72. The process of claim 71, further comprising recovering the remaining starch product after it has been washed.

73. The process of claim 71, wherein the feed starch is from dent corn, waxy corn, high amylose ae genetic corn, potato, tapioca, rice, pea, wheat, waxy wheat, or a combination of two or more thereof.

74. The process of claim 71, wherein the feed starch is heated to at least partially gelatinize the starch prior to treatment with glucanotransferase.

75. The process of claim 71, wherein the feed starch is treated with glucanotransferase in aqueous solution or suspension at a temperature of about 70-100° C. and a pH of about 5.0-8.5.

76. The process of claim 71, wherein the debranching enzyme is isoamylase or pullulanase.

77. The process of claim 71, further comprising membrane filtering a solution or dispersion of the starch product to increase the concentration of amylose fragments that have a degree of polymerization (DP) of at least 35.

78. The process of claim 71, wherein the glucanotransferase is used in a dosage of about 1-18,000 GTU per gram of feed starch.

79. The process of claim 71, wherein the glucanotransferase is used in a plurality of dosages that are supplied to the feed starch at separate times.

80. The process of claim 71, wherein the debranching enzyme is used in a dosage of at least about 0.1 ml per gram of chain extended starch.

81. The process of claim 71, wherein in step (d) the starch product is heated to about 100-150° C. at a moisture content of about 15-35% by weight.

82. The process of claim 81, wherein in step (d) the starch product is heated to about 120-130° C. at a moisture content of about 22-26% by weight.

83. The process of claim 71, wherein the starch product has a total dietary fiber (TDF) value of at least about 10% by weight before the heat moisture treatment in step (d).

84. The process of claim 71, wherein the starch product has a total dietary fiber (TDF) value of at least about 50% by weight after the heat moisture treatment of step (d).

85. The process of claim 71, wherein the starch product has a resistant starch value of at least 40% by weight before the heat moisture treatment of step (d).

86. The process of claim 71, wherein the debranched starch produced in step (b) is gelatinized in a jet cooker to solubilize the starch, and then is cooled to about 20-90° C. to crystallize.

87. The process of claim 71, wherein the starch product has a crystal morphology of A form, B form, or a combination thereof.

88. The process of claim 72, wherein at least about 50% by weight of the recovered starch product has a degree of polymerization (DP) from about 24-100.

89. The process of claim 72, wherein the recovered starch product has a peak melting temperature of greater than about 105° C.

90. The process of claim 72, wherein the recovered starch product is thermally stable in water at temperatures up to at least about 90° C.

91. The process of claim 72, wherein the recovered starch product has an enthalpy as measured by differential scanning calorimetry of at least about 20 Joules/gram.

92. A starch product produced by a process comprising:
(a) treating a feed starch with glucanotransferase to produce a chain-extended starch;
(b) treating the chain-extended starch with a debranching enzyme to produce a starch product that comprises amylose fragments;
(c) crystallizing at least part of the starch product;
(d) heating the starch product in the presence of moisture; and
(e) washing the starch product to remove at least some non-crystallized starch.

93. A food product comprising a starch, wherein the starch is produced by a process comprising:
(a) treating a feed starch with glucanotransferase to produce a chain-extended starch;
(b) treating the chain-extended starch with a debranching enzyme to produce a starch product that comprises amylose fragments;
(c) crystallizing at least part of the starch product;
(d) heating the starch product in the presence of moisture; and
(e) washing the starch product to remove at least some non-crystallized starch.
